(19) United States
(12) Patent Application Publication
(10) Pub. No.: US 2009/0281312 A1
(43) Pub. Date: Nov. 12, 2009

(54) PROCEDURE FOR THE TREATMENT OF PURINES AND RESULTING PRODUCT

(76) Inventors: Josep Tapias Parcerisas, Barcelona (ES); Jaime Dalmau Farnes, Barcelona (ES)

Correspondence Address: GOTTLIEB RACKMAN & REISMAN PC, 270 MADISON AVENUE, 8TH FLOOR, NEW YORK, NY, 10016-0601, US

(21) Appl. No.: 11/916,697

(22) PCT Filed: Jun. 12, 2006
(86) PCT No.: PCT/IB2006/001553

§ 371 Date: Dec. 6, 2007

(30) Foreign Application Priority Data
Jun. 13, 2005 (ES) P 200501414

(51) Int. Cl. C07D 473/00

(52) U.S. Cl. 544/264

(57) ABSTRACT

The procedure for the treatment of purines includes a first phase (1) of electrolytic treatment of the mentioned liquid part in which the metallic nitrates are destroyed by oxidation-reduction and a second phase (2) of physical-chemical treatment of purification and alteration of the properties of the liquid part, eliminating or reducing the pollutants and the CDO; and a third phase (3) in which a substance is added for the use of the resulting product as a base in the manufacture of detergents, medicines and/or cosmetics. The resulting product is sterile, with a total reduction of the metallic nitrates, low nitrogen ammoniacal level, pH between 7.5 to 9.5 and contains phosphorus between 1.5 and 4.5 milligrams/litre, sodium between 750 and 1,800 milligrams/litre, nitrates lower than 1 milligram/litre, ammonia between 2,500 and 4,500 milligrams/litre and its maximum CDO is 5,500 milligrams/litre.

### OBJECT OF THE INVENTION

The present invention refers to a procedure for the treatment of purines allowing for the obtaining of a product whose chemical characteristics allow its use as base or raw material in diverse industrial sectors.

### BACKGROUNDS OF THE INVENTION

At present, the generation of purines is a serious problem for the cattle industry, due to their contaminating characteristics they must be treated adequately. However, no totally effective treatments exist nor do they allow the utilization of the resulting products as raw material in other industries.

The majority of the current treatments are based principally on the physical separation of the solid part from the liquid part, letting the solid part dry for its possible utilization as fertilizer and the more or less partial neutralization of the liquid part in order that its disposal is as least harmful as possible.

It is known that the above mentioned liquid part of the purines includes a high proportion of nitrates, especially metallic nitrates, microbiological activity and a high CDO (Chemical Demand of Oxygen) while, the purines also include other compounds that are usable in diverse chemical industries and even as active elements for dermatological treatments, they cannot be taken advantage of for the existence of the mentioned contaminating elements which accompany them.

### DESCRIPTION OF THE INVENTION

The procedure for the treatment of purines of this invention presents a series of technical particularities that allow the elimination of components or contaminating substances from the purines and to avoid their disposal in the environment by means of the utilization of the beneficial substances to obtain a base product that, by means of a final modification, is usable as raw material in the pharmacological or cosmetics industries, detergents, or as any chemical product where the above mentioned properties are suitable.

The procedure is applied on the liquid part of the purines, obtained by means of the separation from the solid part, which can be designated for conventional use as additive for compost.

The above mentioned liquid part is treated in the first phase of electrolytic treatment in which the metallic nitrates are destroyed by process of oxidation-reduction and in the second procedural physical-chemical phase of purification and alteration of the properties of the above mentioned liquid treated part, eliminating or reducing the pollutants that it contains and reducing the CDO. After these two phases, one proceeds to the adding of a substance which functions as a structural molecule adapted in every case so as to allow the use of the product in the manufacture of certain products, such as detergents, medicines or cosmetics.

The phase of electrolytic treatment is done in a compartmented electrolytic cell in which are contained various electrodes connected to a high frequency generator causing in the above mentioned cell the destruction of the metallic nitrates and the nitrogen being freed in the form of gas. The used electrodes can be of diverse characteristics, including anodes and cathodes of iron, stainless steel, titanium or titanium plate among others.

After the electrolytic phase, it is possible to proceed to filtration for the separation of the primary generated residues, which can be used as additive for fertilizers.

The physical-chemical phase includes the coagulation of the liquid part using organic, inorganic and/or mixed coagulants. After the coagulation, one proceeds to a neutralization of the pH by means of the addition of sodium, calcium and/or magnesium salts reducing notably its level.

Later, one proceeds to a flocculation of the liquid part by means of the use of anionic, cationic and/or not ionic additives.

After these phases, one proceeds to a decantation of the materials in suspension for the separation of the liquid product of those remaining parts in the form of solid residue. This separation of the decanted material is realized by means of filtration using a press filter, for example.

The resulting liquid substance presents as properties that it is colourless, practically odourless and transparent, whilst retaining the biological properties of the purines, with which the aforementioned addition of an additive as structural molecule allows its use as raw material in the production of different products in the fields of cosmetics, pharmaceuticals, detergents etc. The product can be used to substitute water contributing active elements which are appropriate due to their beneficial characteristics for use in products destined for example to dermatology.

The substance or resulting product of the procedure presents basically the following properties:
It is sterile,
It presents a total reduction of metallic nitrates,
It includes a low content of nitrogen ammoniacal
Its pH is between 7.5 and 9.5,
It contains phosphorus between 1.5 and 4.5 mg/l,
It contains sodium between 750 and 1,800 mg/l,
It contains nitrates in a lower quantity than 1 mg/l,
It contains ammonia between 2,500 and 4,500 mg/l,
Its maximum CDO is of 5,500 mg/l.

### DESCRIPTION OF THE FIGURES

To complement the description being undertaken and in order to facilitate the comprehension of the characteristics of the invention, this description is accompanied by a set of drawings in which, with illustrative and not limitative character, the following has been represented:

FIG. 1 shows a scheme of the procedural object of the invention.

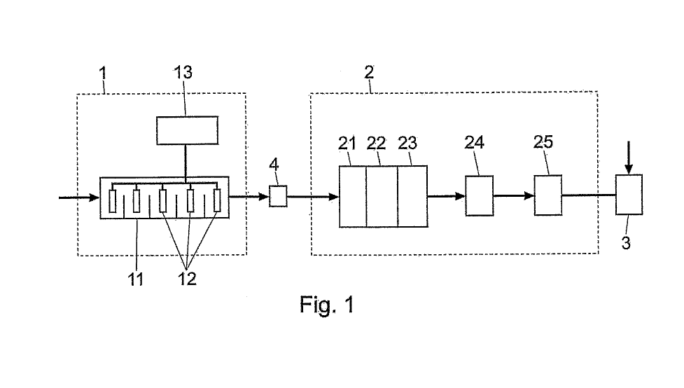

### PREFERRED PROCEDURE OF THE INVENTION

As can be seen in the referenced figures, the procedure includes a phase (1) of electrolytic treatment in which the liquid part of the purines is treated in an electrolytic cell (11) compartmented, whereby various electrodes (12) of iron, stainless steel, titanium and/or titanium plate amongst others are used. These electrodes (12) are connected, as cathodes and anodes, to a high frequency generator (13) for the destruction of the metallic nitrates and freeing nitrogen in gaseous form.

After the electrolytic treatment phase (1) it is possible to undergo the filtration (4) of the primary residues and the resulting product, due to its characteristics, is usable as fertilizer, in which case we pass to the physical-chemical phase (2) of the procedure.

In the above mentioned physical-chemical phase (2) coagulation (21) is done by means of the addition of organic, inorganic and/or mixed coagulants. Later, neutralization (22) of the pH is done by means of the addition of sodium, calcium and/or magnesium salts. Later, one proceeds to a flocculation (23) of the liquid part by means of the addition of anionic, cationic and/or not ionic additives and later to the decantation (24) and filtration (25) by means of a press-filter.

In a third phase (3) of the procedure, one proceeds to the addition of a substance as structural molecule for the use of the resulting product as base or raw material in the manufacture of detergents, medicines, cosmetics, etc.

Having sufficiently described the nature of the invention, as well as an example of preferred usage procedure, notice is given that the materials, shape, size and disposition of the described elements will be able to be modified, only when it does not suppose an alteration of the essential characteristics of the invention claimed hereafter.

1. Procedure for the treatment of purines, which after the physical separation of the solid part from the liquid part is characterized by comprising of: the first phase (1) of electrolytic treatment of the above mentioned liquid part in which the metallic nitrates are destroyed by oxidation-reduction process; optionally the filtration (4) of the liquid part for the separation of the primary residues; re-use as fertilizer, the second phase (2) of physical-chemical purification and alteration of the properties of the liquid treated part, eliminating or reducing the contaminating components that it contains and reducing the CDO (Chemical Demand of Oxygen) and a third phase (3) in which a substance which acts as an adequate structural molecule is added for the use of the resulting product as a base in the manufacture of detergent products, medicines, cosmetics, as well as any other chemical product with adequate characteristics.

2. Procedure, according to claim 1, characterized by the fact that the phase (1) of electrolytic treatment is done in a compartmented electrolytic cell (11) inside which there are various electrodes (12) connected to a high frequency generator (13), operatively suitable for the destruction of the metallic nitrates and freeing of nitrogen in the form of gas.

3. Procedure, according to claim 1, characterized by the fact that the physical-chemical phase (2) comprises:
Coagulation (21) of the liquid part.
Neutralization (22) of the pH.
Flocculation (23) of the liquid part.
Decantation (24) of the materials in suspension.
Filtration (25) until the stable liquid product is obtained.

4. Procedure, according to the claim 1, characterized by the fact that the coagulation (21) is done by means of organic, inorganic and/or mixed coagulants.

5. Procedure, according to the claim 1, characterized by the fact that the neutralization (22) is done by means of sodium, calcium and/or of magnesium salts.

6. Procedure, according to claim 1, characterized by the fact that the flocculation (23) is done by means of anionic, cationic and/or not ionic additives.

7. Procedure, according to claim 1, characterized by the fact that the filtration (25) process is done by means of a press-filter.

9. Procedure, according to claim 1, characterized by the fact that the substance added in phase (3) as structural molecule has detergent, emulsifying and/or moisturizing properties allowing the application of the resulting product as a base of detergent, cosmetic products, pharmaceutical products, etc.

10. Product obtained by the procedure mentioned in the previous claims obtained from the liquid part of purines, characterized by the fact that:
It is sterile,
It presents total reduction of metallic nitrates,
It contains a low nitrogen ammoniacal content,
Its pH is between 7.5 and 9.5,
It contains phosphorus between 1.5 and 4.5 milligrams/litre,
It contains sodium between 750 and 1,800 milligrams/litre,
It contains nitrates at less than 1 milligrams/litre,
It contains ammonia between 2,500 and 4,500 milligrams/litre,
Its maximum CDO is 5,500 milligrams/litre.
